(19) United States
(12) Patent Application Publication
(10) Pub. No.: US 2006/0024824 A1
(43) Pub. Date: Feb. 2, 2006

(54) Magnetic cell separation method

(76) Inventors: Steven Woodside, Vancouver (CA); Terry Thomas, Vancouver (CA)

Correspondence Address: BERESKIN AND PARR, 40 KING STREET WEST, BOX 401, TORONTO, ON, M5H 3Y2, CA

(21) Appl. No.: 11/182,020

(22) Filed: Jul. 15, 2005

Related U.S. Application Data
(60) Provisional application No. 60/588,320, filed on Jul. 16, 2004.

(51) Int. Cl. C12N 5/08

(52) U.S. Cl. 435/366; 424/93.7

(57) ABSTRACT

An improved magnetic target separation method is described. In particular, the magnetic target separation method can be used to separate target entities from non-target entities in a sample contained in a vessel without needing to remove the vessel from the magnetic field gradient.

This application claims priority under 35 USC 119(e) to U.S. Provisional Application No. 60/588,320 filed on Jul. 16, 2004.

### FIELD OF THE INVENTION

The present invention relates to an improved method for separating magnetically labeled target entities in a sample using an applied magnetic field. In particular, the method allows for targeted entities to be separated and washed in a vessel without the need to remove the vessel from the magnetic field gradient.

### BACKGROUND OF THE INVENTION

In many applications it is desirable to enrich, or alternatively deplete, certain target entity populations in a biological sample. For example, the separation of specific cell types from peripheral blood, bone marrow, spleen, thymus and fetal liver is key to research in the fields of hematology, immunology and oncology, as well as diagnostics and therapy for certain malignancies and immune disorders.

Most cell separation techniques require that the input sample be a single cell suspension. For this reason, blood has historically been the most common tissue used for cell separations. Purified populations of immune cells such as T cells and antigen presenting cells are necessary for the study of immune function and are used in immunotherapy. Investigation of cellular, molecular and biochemical processes requires analysis of certain cell types in isolation. Numerous techniques have been used to isolate T cell subsets, B cells, basophils, NK cells and dendritic cells from blood for these investigations.

More recently, enzymatic digestion methods have been developed to dissociate tissues from solid organs into single cell suspensions, permitting distinct cell types to be isolated.

Hematopoietic cells and immune cells have been separated on the basis of physical characteristics such as density and on the basis of susceptibility to certain pharmacological agents which kill cycling cells. The advent of monoclonal antibodies against cell surface antigens has greatly expanded the potential to distinguish and separate distinct cell types. There are two basic conceptual approaches to separating cell populations from blood and related cell suspensions using monoclonal antibodies. They differ in whether it is the desired or undesired cells which are distinguished/labeled with the antibody(ies).

In positive selection techniques, the desired cells are labeled with antibodies and removed from the remaining unlabeled/unwanted cells. In negative selection, the unwanted cells are labeled and removed. Antibody/complement treatment and the use of immunotoxins are negative selection techniques, but FACS sorting and most batch-wise immunoadsorption techniques can be adapted to both positive and negative selection. In immunoadsorption techniques, cells are selected with monoclonal antibodies and preferentially bound to a surface which can be removed from the remainder of the cells, e.g. a column of beads, a flask, or magnetic particles. Immunoadsorption techniques have won favor clinically and in research because they maintain the high specificity of cell targeting with monoclonal antibodies, but unlike FACSorting, they can be scaled up to directly process the large numbers of cells in a clinical harvest and they avoid the dangers of using cytotoxic reagents such as immunotoxins and complement.

Magnetic separation is a process used to selectively retain magnetic materials within a vessel, such as a centrifuge tube or column, in a magnetic field gradient. Targets of interest, such as specific biological cells, proteins and nucleic acids, can be magnetically labeled by binding of magnetic particles to the surface of the targets through specific interactions including immuno-affinity interactions. Other useful interactions include drug-drug receptor, antibody-antigen, hormone-hormone receptor, growth factor-growth factor receptor, carbohydrate—lectin, nucleic acid sequence—complementary nucleic acid sequence, enzyme—cofactor or enzyme-inhibitor binding. The suspension, containing the targets of interest within a suitable vessel, is then exposed to magnetic field gradients of sufficient strength to separate the targets from other entities in the suspension. The vessel can then be washed with a suitable fluid to remove the unlabeled entities, resulting in a purified suspension of the targets of interest.

The majority of magnetic labeling systems use superparamagnetic particles with monoclonal antibodies or streptavidin covalently bound to their surface. In cell separation applications these particles can be used for either positive selection, where the cells of interest are magnetically labeled, or negative selection where the majority of undesired cells are magnetically labeled. Magnetic separation applications where the targets of interest are proteins or nucleic acids would be considered positive selection approaches since the entity of interest is typically captured on the magnetic particle. The diameter of the particle used varies widely from about 50-100 nm for MACS particles (Miltenyi Biotec) and StemSep™ colloid (StemCell Technologies), through 150-450 nm for EasySep® (StemCell Technologies) and Imag (BD Biosciences) particles, up to 4.2 μm for Dynabeads (Dynal Biotech). The type of particle used is influenced by the magnet technology employed to separate the labeled entities.

There are two important classes of magnetic separation technologies, both of which, for convenience and for practical reasons, use permanent magnets as opposed to electromagnets. The first class is column-based high-gradient-magnetic-field separation technology that uses small, weakly magnetic particles to label the targets of interest, and separates these targets in a column filled with a magnetizable matrix. Very high gradients are generated close to the surface of the matrix elements when a magnetic field is applied to the column. The high gradients are necessary to separate targets labeled with these relatively weakly magnetic particles. The second class is tube-based technology that uses more strongly magnetic particles to label the targets of interest. These targets of interest are then separated within a centrifuge-type tube by magnetic field gradients generated by a magnet outside the tube. This method has the advantage that it does not rely on a magnetizable matrix to generate the gradients and therefore does not require an expensive disposable column or a reusable column with an inconvenient cleaning and decontamination procedure.

Once placed within the magnet, targeted cells migrate toward the region or regions of highest magnetic field strength and are retained within the magnetic field while the unlabeled cells are drawn off. The targeted cells can then be collected and used in the desired application after removal from the magnetic field. In the event that negative selection is required, the unlabeled cells are drawn off and can be utilized for a variety of applications such as cell sorting.

Tetrameric antibody complexes (TAC) are comprised of two mouse IgG₁ monoclonal antibodies held in tetrameric array by two rat anti-mouse IgG₁ antibody molecules. EasySep® reagents cross-link magnetizable particles to cells of interest using TAC where one mouse antibody targets the particles and the other targets surface markers on the cells of interest. The EasySep® magnet then separates the magnetically labeled cells from non-labeled cells within a standard centrifuge tube.

In tube-based systems such as Dynal and EasySep®, the separation vessel is typically a standard centrifuge tube held in a magnetic field, and the region or regions of highest magnetic field strength are along the walls of the tube. Thus, magnetically labeled cells migrate through the suspending fluid and collect at the walls of the tube. After this migration takes place, the suspending fluid and suspended non-target cells, otherwise referred to as the supernatant, can be removed. This sequence of steps enriches the target cells by preferentially retaining the targeted cells in the tube. To further improve the enrichment, the retained cells which include targeted cells and non-specifically retained non-target cells can be resuspended in fluid and separated again. Prior art teaches that for efficient positive selection enrichment in tube-based magnetic separation, the tube containing the retained target cells should be removed from the magnet after supernatant removal but before resuspension. It is assumed that this allows all the cells retained during the magnetic separation to be completely resuspended and removed from the wall of the separation vessel and dispersed in fresh fluid, thereby providing initial conditions for the subsequent magnetic separation step that are similar to the previous separation. In this case an action is required to remove the applied magnetic field. Although this extra step is relatively simple for a separation performed manually, for automated separations it requires an additional mechanism, and therefore additional complexity. Accordingly it would be desirable to have a method where the cells can be resuspended within the applied magnetic field used for magnetic separation.

### SUMMARY OF THE INVENTION

The present invention relates to an improved method for separating target entities using magnetic separation. Accordingly, the present invention provides a method of magnetic separation of target entities from a sample containing a mixture of target entities and non-target entities suspended in a fluid, with said method comprising:
a. labeling said target entities with magnetic particles wherein said entities are suspended in a fluid and said fluid is contained in a vessel;
b. placing said vessel in a magnetic field gradient of sufficient strength to separate the magnetically labeled target entities from the non-target entities, wherein the magnetically labeled target entities are separated by moving to the walls of the vessel;
c. removing the fluid containing non-target entities from the vessel after the magnetically labeled entities are separated, the vessel being maintained in the magnetic field gradient;
d. adding fluid to the vessel being maintained in the magnetic field gradient and mixing; and
e. removing the fluid containing the non-target entities from the vessel being maintained in the magnetic field gradient after the magnetically labeled entities are separated.

The method optionally further comprises repeating steps d and e as required to obtain the desired purity of the target entities.

In another embodiment, the method further comprises collecting the fluid containing the non-target entities removed in steps c and e.

In yet another embodiment, the method is performed by an automated instrument that automatically performs steps a-e, and also optionally repeats steps d and e as required to obtain the desired purity of the target entities and/or automatically collects the fluid containing the non-target entities removed in steps c and e.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present inventors have determined that in a target separation method using magnetic separation, the sample does not need to be removed from the magnetic field after supernatant removal and prior to resuspending the target entity during washing steps. The inventors have shown that leaving the sample in the magnetic field does not reduce the efficiency of the resuspension step. In particular, the purity and recovery levels of cells were comparable to levels achieved when cells were resuspended outside of the magnetic field.

Methods of the Invention:

The method of the invention is an improvement over the existing method as it eliminates the need for the extra step of removing the sample from the magnetic field gradient which is especially advantageous for automated separation methods.

Accordingly, the present invention provides a method of magnetic separation of target entities from a sample containing a mixture of target entities and non-target entities suspended in a fluid, with said method comprising:
a. labeling said target entities with magnetic particles wherein said entities are suspended in a fluid and said fluid is contained in a vessel;
b. placing said vessel in a magnetic field gradient of sufficient strength to separate the magnetically labeled target entities from the non-target entities, wherein the magnetically labeled target entities are separated by moving to the walls of the vessel;
c. removing the fluid containing non-target entities from the vessel after the magnetically labeled entities are separated, the vessel being maintained in the magnetic field gradient;
d. adding fluid to the vessel being maintained in the magnetic field gradient and mixing; and
e. removing the fluid containing the non-target entities from the vessel being maintained in the magnetic field gradient after the magnetically labeled entities are separated.

The method optionally further comprises repeating steps d and e as required to obtain the desired purity of the target entities.

In another embodiment, the method further comprises collecting the fluid containing the non-target entities removed in steps c and e.

In yet another embodiment, the method is performed by an automated instrument that automatically performs steps a-e, and also optionally repeats steps d and e as required to obtain the desired purity of the target entities and/or automatically collects the fluid containing the non-target entities removed in steps c and e.

The sample can be any sample containing the desired target entities and non-target entities including, but not limited to, blood samples such as whole blood and processed blood, tissue samples, bone marrow, pleural and peritoneal effusions, homogenized tumor samples, or leukapheresis samples from patients receiving mobilizing growth factors. In one embodiment, the sample is mouse spleen or density gradient separated human blood. In another embodiment, the sample is in the form of a cell suspension.

The fluid can be any fluid that is free of non-target entities. In one embodiment, the fluid is an aqueous solution, such as saline or cell culture medium.

The vessel can be any vessel that is amenable for use in a magnetic separation method including, but not limited to, tubes (such as centrifuge tubes), flasks, plates, syringes, bottles, pipettes or culture dishes. The magnetic particles can be any magnetic particle that can be used in a magnetic separation method. Suitable magnetic particles include particles in ferrofluids, other colloidal magnetic solutions and particles in suspension. “Ferrofluid” refers to a colloidal solution containing particles consisting of a magnetic core, such as magnetite (Fe₃O₄) coated or embedded in material that prevents the particle cores from interacting. Likewise, magnetic particles in suspension consist of magnetic cores coated or embedded in material that prevents the particle cores from interacting. Examples of such materials include proteins, such as ferritin, polysaccharides, such as dextrans, or synthetic polymers such as sulfonated polystyrene cross-linked with divinylbenzene. The core portion is generally too small to hold a permanent magnetic field. The ferrofluids become magnetized when placed in a magnetic field. Examples of ferrofluids and methods for preparing them are described by Kemshead J. T. (1992) in J. Hematotherapy, 1:35-44, at pages 36 to 39, and Ziolo et al. Science (1994) 257:219 which are incorporated herein by reference. Particles of dextran-iron complex or dextran-coated iron oxides are preferably used in the process of the invention. (See Molday, R. S. and McKenzie, L. L. FEBS Lett. 170:232, 1984; Miltenyi et al., Cytometry 11:231, 1990; and Molday, R. S. and MacKenzie, D., J. Immunol. Methods 52:353, 1982; Thomas et al., J. Hematother. 2:297 (1993); and U.S. Pat. Nos. 4,452,733, 5,698,271 and 5,512,332 and German Patent No DE196 24 426A1 which are each incorporated herein by reference).

In one embodiment, the magnetic particles have a mean diameter of less than 5 μm. In another embodiment, the magnetic particles have a mean diameter of less than 2 μm. In yet another embodiment, the magnetic particles have a mean diameter of less than 1 μm. In yet another embodiment, the magnetic particles have a mean diameter of less than 500 nm. In a specific embodiment, the magnetic particles are magnetic nanoparticles that have a mean diameter of about 160 nm. The mean particle diameter may be measured in a number of ways depending on the expected size of the particles as is understood by those skilled in the art.

Magnetic particles used for cell magnetic separation are more correctly magnetizable particles, in that they are magnetic in the presence of a magnetic field and non-magnetic in the absence of a magnetic field. Such particles are termed paramagnetic or superparamagnetic depending on the material. Particles with more magnetizable material experience a stronger magnetic force when exposed to a magnetic field gradient than particles with less material because the force is proportional to the volume of magnetizable material. In general then, it is necessary to use a stronger magnetic field gradient to separate cells labeled with smaller particles. Conversely, it is also generally possible to separate cells faster using larger particles because the greater force exerted on the labeled cells moves them more rapidly through the suspending fluid towards the attracting magnetic pole.

In positive selection using a tube-based system, one round of separation consists of separation in the magnet, supernatant removal and resuspension of the remaining cells. The number of rounds of separation performed will influence the purity and recovery of the target cells. Each round of separation preferentially removes unlabelled or relatively weakly labeled cells, and thus improves purity. Each round of separation also results in the loss of some target cells and therefore lower recovery. The purity improvement and loss of recovery are highly dependent on the type of cell being targeted for enrichment and the parameters of the separation such as time in the magnet for each round of separation, incubation times for the labeling reagents, and labeling specificity and quantity for the magnetic particle.

The target entities can be any entity that one wishes to separate using magnetic separation, such as cells, cell organelles, proteins or nucleic acid sequences. The target entities are preferably cells including, but not limited to, T cells, B cells, basophils, NK cells, dendritic cells, tumor cells, stem cells, hematopoietic progenitor cells, monocytes, mesenchymal cells, erythrocytes, mammary epithelial cells, eosinophils, neural cells, endothelial stem cells, endothelial progenitor cells or embryonic stem cells.

The target entities, preferably cells, can be labeled with the magnetic particles using antibodies with specificity for the target entities. In one embodiment, the antibodies that bind to the target entities may be chemically bound to the surface of the magnetic particles, for example, using cyanogen bromide. In another embodiment, the magnetic particles may be coated with a second antibody having specificity for the antibodies that bind to the target entity.

In a particular embodiment, the target entities are bound to the magnetic particles using tetrameric antibody complexes (TACs). A tetrameric antibody complex may be prepared by mixing a first monoclonal antibody which is capable of binding to at least one antigen on the surface of the magnetic particle, and a second monoclonal antibody that binds to the target entity. The first and second monoclonal antibodies are from a first animal species. The first and second antibody are reacted with an about equimolar amount of monoclonal antibodies of a second animal species which are directed against the Fc-fragments of the antibodies of the first animal species. The first and second antibody may also be reacted with an about equimolar amount of the F(ab′)2 fragments of monoclonal antibodies of a second animal species which are directed against the Fc-fragments of the antibodies of the first animal species. (See U.S. Pat. No. 4,868,109 to Lansdorp, which is incorporated herein by reference for a description of tetrameric antibody complexes and methods for preparing same).

In accordance with the magnetic separation method, the sample containing the target cells to be recovered, is reacted with a tetrameric antibody complex, so that the antibody reagents bind to the target cells present in the sample to form cell conjugates of the target cells, and the antibody reagents. The reaction conditions are selected to provide the desired level of binding of the target cells and the antibody reagents. Preferably the sample is incubated with the antibody reagents for a period of 0 to 60 minutes, preferably 15 minutes, at between 0° C. and 40° C., or preferably between 4° C. and ambient room temperature. The concentration of the antibody reagents is selected depending on the estimated concentration of the targeted differentiated cells in the sample. Generally, the concentration is between about 0.1 to 50 μg/ml of sample. The magnetic particles are then added and the mixture is incubated for a period of about 5 minutes to 60 minutes, preferably 10 min at the selected temperature. The sample is then ready to be separated with a magnet that generates a magnetic field in its interior cavity that has a strong enough magnetic gradient to separate target entities labeled with the magnetic particles without the additional magnetic field gradients provided by a column matrix. The magnetically labeled target entities will migrate to the surface of the tube. The labeled target entities are then separated from the non-target entities in a series of wash steps. Generally, after the magnet is applied, the magnet and the tube are inverted and the supernatant fraction is poured off. Alternately, the supernatant is removed using a pipette. A fluid, such as culture medium, is then added to the tube and the cell suspension is mixed and set aside to separate magnetically a second time. The tube is then inverted again to pour off the supernatant and the wash step is repeated until the target entities reach the desired level of purity. The percentage purity and recovery of the target entities can be determined by various methods including flow cytometry.

Applications:

Separation methods of the present invention are of particular benefit to the study of pluripotent stem cells and tissue-specific stem cells from adults. The rapidly growing field of stem cell research is spurred by the potential of these cells to repair diseased or damaged tissues. Bone marrow (hematopoietic) stem cells were the first adult stem cells to be purified and used clinically and the therapeutic potential of hematopoietic stem cells is now well documented. Transplantation of hematopoietic cells from peripheral blood and/or bone marrow is increasingly used in combination with high-dose chemo- and/or radiotherapy for the treatment of a variety of disorders including malignant, non-malignant and genetic disorders. Very few cells in such transplants are capable of long-term hematopoietic reconstitution, and thus there is a strong stimulus to develop techniques for purification of hematopoietic stem cells. Accordingly, in one embodiment, the invention provides a use of the target entities purified from the methods of the invention for the treatment of malignant, non-malignant or genetic disorders. In another embodiment, the invention provides a method for treating malignant, non-malignant or genetic disorders comprising administering an effective amount of the target entities purified from the methods of the invention to an animal in need thereof.

Epithelial cancers of the bronchi, mammary ducts and the gastrointestinal and urogenital tracts represent a major group of solid tumors seen today. Micrometastatic tumor cell migration is thought to be an important prognostic factor for patients with epithelial cancer (Braun et al., 2000; Vaughan et al., 1990). The ability to detect such metastatic cells is limited by the effectiveness of tissue or fluid sampling and the sensitivity of tumor detection methods. A technique to enrich circulating epithelial tumor cells in blood samples would increase the ability to detect metastatic disease and facilitate the study of such rare cells to determine the biological changes which enable spread of the disease. Accordingly, in one embodiment, the invention provides a use of the target entities purified from the methods of the invention for the detection of cancer and metastatic disease. In another embodiment, the invention provides a method for detecting cancer and metastatic disease in a biological sample comprising detecting the target entities purified from the methods of the invention. For example, stem cell positive selection based on CD34 or CD133 surface antigen expression can be used to remove tumor cells that don't express CD34 or CD133.

Furthermore, serious complications and indeed the success of a transplant procedure is to a large degree dependent on the effectiveness of the procedures that are used for the removal of cells in the transplant that pose a risk to the transplant recipient. Such cells include T lymphocytes that are responsible for graft versus host disease (GVHD) in allogeneic grafts, and tumor cells in autologous transplants that may cause recurrence of the malignant growth. Accordingly, in one embodiment, the invention provides a use of the fluid containing the non-target entities produced by the methods of the invention for generating a cell population deplete of T-cells. It is also important to debulk the graft by removing unnecessary cells and thus reducing the volume of cryoprotectant to be infused. Accordingly, in another embodiment, the invention provides a use of the fluid containing non-target entities produced by the methods of the invention for generating a graft deplete of unnecessary cells.

In certain instances it is desirable to remove or deplete tumor cells from a biological sample, for example in bone marrow transplants. Accordingly, the invention provides a use of the fluid containing non-target entities produced by the methods of the invention for generating a bone marrow cell population deplete of tumor cells.

The following non-limiting examples are illustrative of the present invention:

### EXAMPLES

### Example 1

### Magnetic Labelling and Separation of Cells with Dynal Reagents

Human Cells

The procedure outlined below was used for direct selection of CD19+cells from a sample of Human Peripheral Blood Mononuclear Cells (PBMCs) using 4.5 μm mean diameter “Dynabeads M-450 CD19” from Dynal. In this procedure, human PBMCs were incubated with Dynabeads, which are conjugated to anti-Human CD19 antibodies, thus magnetically labelling the CD19+ cells in the sample. These cells were then separated from the rest of the cell suspension using 4 different magnets with a range of magnetic field strengths.
Strong: Dynal Magnetic Particle Concentrator (MPC)
Medium strength: Small bar magnet
Weak: Common cabinet clip magnet (round)
Very weak: Common refrigerator magnet (round).

A flat refrigerator magnet was assessed as well, but the particles settled out of solution faster than moving toward the magnet, so no separation was attainable. Results were not collected for the latter magnet.

Purity of selected cells and of the target cells lost in the depleted fraction was measured using expression of the CD20 antigen. Recovery of target cells was calculated from the number of total cells and the CD20+ cell purity in the start and enriched sample. A total of eight conditions were tested:
A) Separation in strong magnet, resuspension (washing) inside the magnetic field
B) Separation in strong magnet, resuspension (washing) outside the magnetic field
C) Separation in medium magnet, resuspension (washing) inside the magnetic field
D) Separation in medium magnet, resuspension (washing) outside the magnetic field
E) Separation in weak magnet, resuspension (washing) inside the magnetic field
F) Separation in weak magnet, resuspension (washing) outside the magnetic field
G) Separation in very weak magnet, resuspension (washing) inside the magnetic field
H) Separation in very weak magnet, resuspension (washing) outside the magnetic field
   Procedure:
1. Prepared a single cell suspension of human peripheral blood mononuclear cells by density gradient separation at a concentration of 2.5×10⁷ cells/mL in buffered cell culture medium (PBS+2% Fetal Bovine Serum+1 mM EDTA)
2. Prepared Dynal buffer: PBS+0.1% BSA+2 mM EDTA.
3. Washed M-450 CD19 Dynabeads with Dynal buffer, resuspended in Dynal buffer to their original concentration (4×10⁸ beads/mL).
4. Labeled PBMCs in bulk: Added 250 μL beads to a 10 mL sample of PBMCs at 2.5×10⁷ cells/mL (25 μL beads per mL sample), incubated 20 min on ice (˜2-8° C.) with gentle mixing to prevent beads from settling.
5. Aliquoted 8 mL of labelled cells into eight tubes, 1.0 mL (2.5×10⁷ cells) per tube. Placed tube into magnet for separation #1: Incubation of 2 minutes for the strong magnet (samples A and B), medium strength magnet (samples C and D) and weak magnet (samples E and F) and incubation of 3 minutes for very weak magnet.
6. Gently poured off supernatant, (turned magnet completely upside down), saved this “depleted fraction” for analysis.
7. Resuspended cells retained in tube to a final volume of 1.0 mL with Dynal buffer, gently pipetted up and down to mix. Replaced tube in magnet and incubated for 1 min (or 1.5 min for very weak magnet), poured supernatant into “depleted fraction” tube. Dependent on conditions, the resuspension was done either inside the magnetic field—leaving the tube attached to the magnet, or outside the magnetic field—removing the tube from the magnet before adding buffer.
8. Repeated Step 7 three more times (total 4 washes, 5 separations)
9. After final separation, added 500 μL Dynal buffer for a total volume of about 600 μL due to some retention of buffer from the supernatant pour-off step.
10. Performed a cell count and FACS analysis on enriched cells to determine CD19+ content (Dynabeads not detached).

### Example 2

### Magnetic Labelling and Separation of Cells with EasySep® Reagents

The detailed manual EasySep® procedure is laid out below. In this procedure, the tube containing the labeled cells is removed from the magnet used for separation during the resuspension/wash step (step 6). Easysep® particles are approximately 160 nm in diameter.
1. TAC were comprised of two mouse IgG₁ monoclonal antibodies held in tetrameric array by two rat anti-mouse IgG₁ antibody molecules. EasySep® reagents cross-linked magnetizable particles to cells of interest using TAC where one mouse antibody targeted the particles and the other targeted surface markers on the cells of interest. The EasySep® magnet then separated the magnetically labeled cells from non-labeled cells within a standard centrifuge tube. Prepared nucleated cell suspension at a concentration of 1×10⁸ cells/mL in buffered cell culture medium (e.g. PBS+2% Fetal Bovine Serum+1 mM EDTA). Cells were placed in a 12×75 mm polystyrene tube to properly fit into the EasySep® Magnet.
2. Added EasySep® Positive Selection Cocktail at 100 μL/mL cells. Mixed well and incubated at room temperature for 15 minutes.
3. Mixed EasySep® Magnetic Nanoparticles to ensure that they were in a uniform suspension. Added the particles at 50 μL/mL cells. Mixed well and incubated at room temperature for 10 minutes.
4. Brought the cell suspension to a total volume of 2.5 mL by adding buffered cell culture medium. Mixed the cells in the tube by gently pipetting up and down 2-3 times. Removed the cap from the tube and placed the tube into the magnet. It was set aside for five minutes.
5. Picked up the magnet, and in one continuous motion inverted the magnet and tube, pouring off the supernatant fraction. The magnetically labeled cells remained inside the tube, held by the magnetic field of the EasySep® magnet. Left the magnet and tube in inverted position for 2-3 seconds, then returned to upright position.
6. Removed the tube from the magnet and added 2.5 mL buffered cell culture medium. Mixed the cell suspension by gently pipetting up and down 2-3 times. Placed the tube back in the magnet and was set aside for five minutes.
7. Repeated Steps 5 and 6, and then Step 5 once more, for a total of three 5-minute separations in the magnet. Removed the tube from the magnet and resuspended cells in an appropriate amount of cell culture medium. The positively selected cells were now ready for use.

### Example 3

### Magnetic Labelling and Separation of Cells with BD Imag™ Reagents

1. Prepared a single-cell suspension from the lymphoid tissue of interest according to standard laboratory procedures. Removed clumps of cells and/or debris by passing the suspension through a 70-μm nylon cell strainer. Resuspended cells to 1×10⁸ cells/mL in buffer (PBS+2% FBS+1 mM EDTA).
2. Divided into 5 samples of 5×10⁷ cells.
3. Vortexed the BD Imag particles and added 50 μl of particles for every 107 total cells or 250 uL of particles for the given samples.
4. Mixed thoroughly and incubated for 20 minutes at room temperature.
5. Brought the total volume up to 5 mL, placed the tube on the BD IMagnet or EasySep® magnet and incubated at room temperature for 6 minutes.
6. For the tubes in the BD Imagnet carefully aspirated off the supernatant. For the tubes in the EasySep® magnets poured off the supernatant. The supernatant contained the negative fraction.
7. Added 5 mL of buffer with the tube in the magnet or after removing the tube from the magnet depending on the test conditions. Gently resuspended cells by pipetting up and down, and then returned the tube to the magnet for another 4 minutes.
8. With the tube on the BD Imagnet, carefully aspirated off the supernatant and discard.
9. Repeated Steps 7 and 8 such that a total of 3 rounds of magnetic separation had been performed (6 min, 4 min and 4 min).
10. After the final wash step, resuspended the positive fraction in 1 mL of EasySep® buffer.

### Example 4

### Method for Automated Magnetic Separation of Cells Labelled with EasySep® Reagents

The manual protocol given in Example 2 was modified for automated separation using a prototype instrument as described below. The instrument was a fluid handling robot that used disposable 1 mL tips to add a cocktail of antibodies (step 2) and particles (step 3), and 5 mL tips to add buffer (steps 4 and 6), or mix cells (steps 2, 3, 4 and 6) and transfer cells from one tube to another (step 4, 5). The cells were prepared manually (step 1) and placed on the instrument in a 14 mL tube. Sufficient buffered cell culture medium, positive selection cocktail and well mixed magnetic nanoparticles were placed on the instrument in the correct locations. An empty 14 mL tube was placed in the magnet on the instrument that was used for the magnetic separation steps. A 50 mL tube was also placed on the instrument to receive the supernatant from the separation steps and a pipette tip rack loaded with the correct disposable pipette tips was placed on the instrument.
1. Prepared nucleated cell suspension at a concentration of 1×10⁸ cells/mL in buffered cell culture medium (e.g. PBS+2% Fetal Bovine Serum+1 mM EDTA). Cells were placed in a 17×100 mm polystyrene tube to fit on the automated separator.
2. Instrument added EasySep® Positive Selection Cocktail to cells at 100 μL/mL cells. Instrument mixed cells in the tube by pipetting up and down 3 times and then waited for a 15 minute incubation at room temperature.
3. Instrument added magnetic particles to cells at 50 μL/mL cells. Instrument mixed cells in the tube by pipetting up and down 3 times and then waited for a 10 minute incubation at room temperature.
4. Instrument brought the cell suspension to a total volume of 10 mL by adding buffered cell culture medium. Instrument mixed the cells in the tube by pipetting up and down 3 times. Instrument transferred the cell suspension to the 14 mL tube in the magnet and waited for five minutes to allow magnetic separation.
5. Instrument pipetted off the supernatant fraction from tube in magnet and transferred it to the designated 50 mL tube. The magnetically labeled cells remained inside the tube, held by the magnetic field of the EasySep® magnet.
6. Instrument added 10 mL buffered cell culture medium and mixed the cell suspension by pipetting up and down 3 times then waited for five minutes to allow magnetic separation.
7. Instrument repeated Steps 5 and 6, and then Step 5 once more, for a total of three 5-minute separations in the magnet. We removed the tube from the magnet and resuspended cells in an appropriate amount of cell culture medium.

### Example 5

### Effect of Magnetic Field Used for Separation On Washing of Cells Labelled with EasySep® Reagents

This example shows that the presence of the magnetic field used to magnetically separate the cells does not reduce the efficiency of the resuspension step. EasySep® particles used in this example had a mean diameter of about 160 nm.

CD3+ cells were magnetically labeled using a CD3 positive selection cocktail and dextran coated magnetic nanoparticles as described in steps 1-3 of Example 2. The cells were then divided into samples A and B of equal volumes (X mL). Sample A was separated magnetically as described in steps 4-7 of Example 2 with the cells resuspended between separations with the tube removed from the magnet. The purity of the cells separated from Sample A as assessed by flow cytometry was 99.2% CD3+, with a recovery of 64% of the CD3+ cells present in the sample prior to separation. Sample B was separated magnetically as in steps 4-7 of Example 2 with the modification that the tube was left in the magnet for the resuspension step, step 6. The purity of the cells separated from Sample B, as assessed by flow cytometry, was 99.6% CD3+, with a recovery of 64% of the CD3+ cells present in the sample prior to separation. If the magnetic field were to prevent adequate resuspension of cells, particularly unlabelled cells, then one would expect to see a lower purity in the sample resuspended in the magnet. Because this lower purity is not observed, this example shows that the presence of the magnetic field used to magnetically separate the cells does not reduce the efficiency of the resuspension step. It is therefore acceptable to resuspend the cells in the magnetic field.

### Example 6

### Effect of Magnetic Field Used for Separation On Washing of Cells Labelled with BD Imag™ Reagents

This example shows that the presence of the magnetic field used to magnetically separate the cells does not reduce the efficiency of the resuspension step. The Imag particles used in this example had a mean diameter of approximately 250 nm.

At least 5×10⁸ mouse spleen cells were obtained and split into samples A and B of 2×10⁸ cells each for magnetic labeled using Imag anti-mouse CD8a Particles DM (Sample A) and Imag anti-mouse B220 Particles DM (Sample B) from Becton-Dickinson as described in steps 1 to 4 of Example 3. Samples C and D of 5×10⁷ cells each were set aside for automated separation. Samples A and B were then further divided into 4 samples A1 through A4 and 4 samples B1 through B4 of 5×10⁷ cells each. Samples A1 and B1 were separated magnetically using the Imag magnet as described in steps 5 to 9 of Example 3 with the tube removed from the magnet for the cells resuspension steps. Samples A2 and B2 were separated magnetically using the Imag magnet as described in steps 5 to 9 of Example 3 with the tube in the magnet for the cells resuspension steps. Samples A3 and B3 were separated magnetically using the EasySep® magnet as described in steps 5 to 9 of Example 3 with the tube removed from the magnet for the cells resuspension steps. Samples A4 and B4 were separated magnetically using the EasySep® magnet as described in steps 5 to 9 of Example 3 with the tube in the magnet for the cells resuspension steps. Samples C and D were separated magnetically using Imag anti-mouse CD8a Particles DM (Sample C) and Imag anti-mouse B220 Particles DM (Sample D) from Becton-Dickinson and the EasySep® magnet on the automated instrument as described in Example 4 but with the sample volume, reagent addition volumes, incubation times and magnetic separations times as used for the manual separations in Example 3. The purity of the cells was assessed by flow cytometry and the recovery was calculated based on the number of cells in the initial and separated samples. The purity and recovery for these separations are presented in Tables 1 and 2. If the magnetic field were to prevent adequate resuspension of unlabelled cells after magnetic separation, then one would expect to see a lower purity in the samples resuspended in the magnet. Because this lower purity is not observed, this example shows that the presence of the magnetic field used to magnetically separate the cells does not reduce the efficiency of the resuspension step. It is therefore acceptable to resuspend the cells in the magnetic field.

### Example 7

### Effect of Magnetic Field on Washing of Cells Labelled with Dynal Reagents

Human Cells:

CD19+ human cells were labelled for magnetic separation as described in Example 1 steps 1-5. The cells were then divided into 1 mL samples A through H for separation as described in Example 1 steps 6-10 with specific conditions as follows:
A) Separation in strong magnet, resuspension (washing) inside the magnetic field
B) Separation in strong magnet, resuspension (washing) outside the magnetic field
C) Separation in strength medium magnet, resuspension (washing) inside the magnetic field
D) Separation in strength medium magnet, resuspension (washing) outside the magnetic field
E) Separation in weak magnet, resuspension (washing) inside the magnetic field
F) Separation in weak magnet, resuspension (washing) outside the magnetic field
G) Separation in very weak magnet, resuspension (washing) inside the magnetic field
H) Separation in very weak magnet, resuspension (washing) outside the magnetic field

In each of the above conditions the labelled cells could be seen by the naked eye to move towards the magnet after placing the sample in the magnetic field near the magnet. The time for most of the cells to visibly move to the magnet depended on the strength of the magnet. Using the strong magnet (Dynal MPC) the cells visibly separated in about 2 seconds or less. Using the medium strength magnet, the labelled cells visibly separated in about 15 seconds. Using the weak magnet, the labelled cells visibly separated in about 30 seconds. Using the very weak magnet, the labelled cells visibly separated in about 60 seconds.

The data in Table 5 show that using any given magnet the purity is slightly lower for the samples resuspended in the magnet, but that the difference is not great for the stronger magnets and only becomes significant for the weakest magnet. These data show that high purity and recovery can be obtained using the 4.5 μm diameter Dynabeads when resuspending the labelled cells in the magnet, contrary to the suggested procedure.

### Example 8

### Automated Magnetic Separation of Human Cells Labelled with EasySep® Reagents

This example shows that resuspension in the magnet is effective and eliminates a step that is difficult to automate.

Human nucleated cell suspensions were prepared as described in step 1 of example 4 and sub-divided into samples A and B of approximately 1 mL each, or about 1×10⁸ cells per sample. Sample A was separated manually using the protocol outlined in Example 2, while Sample B was separated with the instrument using the protocol outlined in Example 4. The EasySep® selection cocktail used in each set of comparisons was one of Human CD3, Human CD4, Human CD8, Human CD14 or Human CD19 positive selection cocktail. The results, summarized in Table 3, show the purity of the target cells in the enriched fraction as determined by flow cytometry, and the percentage of the target cells present in the initial sample recovered in the enriched fraction. Where there is more than one comparison with the same selection cocktail, the average over the comparisons is shown ±1 standard deviation.

### Example 9

### Automated Magnetic Separation of Mouse Cells Labelled with EasySep® Reagents

The manual protocol given in Example 2 and the automated protocol give in Example 4 were modified for positive selection of mouse cells to first add PE-conjugated antibodies to the cells. For the manual separation protocol the following steps were performed between steps 1 and 2 in Example 2:
A. Added Mouse FcR Blocker at 10 μL/mL cells and mixed.
B. Added PE labeling reagent and mixed, incubated for 15 min at room temperature.
   For the automated protocol the following steps were performed between steps 1 and 2:
C. Added Mouse FcR Blocker at 10 μL/ml cells and mixed.
D. Instrument added EasySep® PE labeling reagent to cells at 15 μL/mL cells. Instrument mixed well and incubated at room temperature for 15 minutes.
   The selection cocktail for mouse positive selection was the EasySep® PE selection cocktail.

MNC suspensions were prepared from mouse spleen cells and sub-divided into samples A and B of approximately 2 mL each, or about 1×10⁸ cells per sample. Sample A was separated manually using the protocol outlined in Example 2, while Sample B was separated with the instrument using the protocol outlined above. The EasySep® selection cocktail used in each set of comparisons was either Mouse CD19 or Mouse panNK positive selection cocktail. The results, summarized in Table 4, show the purity of the target cells in the enriched fraction as determined by flow cytometry, and the percentage of the target cells present in the initial sample recovered in the enriched fraction. Where there is more than one comparison with the same target cell, the average over the comparisons is shown ±1 standard deviation.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Comparison of cell purity and recovery after magnetic separation
of mouse CD8+ cells from a spleen cell suspension
using 250 nm mean diameter Imag DM particles.

| | % CD8+ of | % recovery |
|---|---|---|
| Sample | viable | CD8 |
| A1 - Resuspend out of IMAG magnet | 97.9 | 14 |
| A2 - Resuspend in IMAG magnet | 97.3 | 17 |
| A3 - Resuspend out of EasySep ® magnet | 96.3 | 50 |
| A4 - Resuspend in EasySep ® magnet | 98.3 | 64 |
| C - Automated separation | 99.4 | 46 |

TABLE 2
Comparison of cell purity and recovery after magnetic separation
of mouse B220+ cells from a spleen cell suspension
using 250 nm mean diameter Imag DM particles.

| | % B220+ of | % recovery |
|---|---|---|
| Sample | viable | B220+ |
| B1 - Resuspend out of IMAG magnet | 99.6 | 35 |
| B2 - Resuspend in IMAG magnet | 97.4 | 41 |
| B3 - Resuspend out of EasySep ® magnet | 98.3 | 110 |
| B4 - Resuspend in EasySep ® magnet | 98.2 | 82 |
| D - Automated separation | 99.5 | 76 |

TABLE 3
Comparison of purity and recovery of various target human cells
(CD3+, CD4+, CD8+, CD14+ and CD19+ cells) obtained
for manual and automated cell separation procedures.

| | | Sample A (Manual) | | Sample B (Instrument) | |
|---|---|---|---|---|---|
| Target Cell | n | % Purity | % Recovery | % Purity | % Recovery |
| CD 3 | 2 | 99.6 ± 0.2 | 64.6 ± 20.6 | 99.7 ± 0.2 | 44.8 ± 14.2 |
| CD 4 | 1 | 99.3 | 42.9 | 99.4 | 35.5 |
| CD 8 | 3 | 96.6 ± 2.8 | 49.4 ± 22.4 | 98.4 ± 1.7 | 52.9 ± 21.4 |
| CD 14 | 1 | 99.1 | 51.6 | 99.5 | 38.4 |
| CD 19 | 5 | 97.5 ± 1.6 | 65.0 ± 19.4 | 98.5 ± 1.0 | 43.5 ± 15.6 |

TABLE 4
Comparison of purity and recovery of CD19+ and panNK+ mouse
cells for manual and automated cell separation procedures

| | | Sample A (Manual) | | Sample B (Instrument) | |
|---|---|---|---|---|---|
| Target Cell | n | % Purity | % Recovery | % Purity | % Recovery |
| CD19 | 1 | 99.7 | 19.8 | 99.6 | 32.0 |
| panNK | 1 | 93.0 | 17.0 | 92.2 | 19.5 |

TABLE 5
Comparison of CD19+ purity and recovery after magnetic
separation with magnets of different field strengths and
resuspension inside or outside of magnetic field.

| | % CD20+ of | % Recovery | % CD20+ |
|---|---|---|---|
| Human CD19+ cells | Viable | CD20+ | in Depleted |
| Start - 2.5 × 10\hat{ }7 cells | 8.21 | | |
| A) Strong magnet; | 96.60 | 68.24 | 2.73 |
| Resuspend inside field | | | |
| B) Strong magnet; | 97.86 | 57.21 | 3.98 |
| Resuspend outside field | | | |
| C) Medium magnet; | 94.83 | 72.08 | 2.90 |
| Resuspend inside field | | | |
| D) Medium magnet; | 98.05 | 45.91 | 3.70 |
| Resuspend outside field | | | |
| E) Weak magnet; | 93.34 | 61.39 | 2.60 |
| Resuspend inside field | | | |
| F) Weak magnet; | 97.72 | 46.47 | 3.77 |
| Resuspend outside field | | | |
| G) Very weak magnet; | 78.97 | 46.17 | 3.40 |
| Resuspend inside field | | | |
| H) Very weak magnet; | 95.42 | 18.22 | 4.87 |
| Resuspend outside field | | | |

We claim:

1. A method of magnetic separation of target entities from a sample containing a mixture of target entities and non-target entities suspended in a fluid, with said method comprising:
a) labeling said target entities with magnetic particles wherein said entities are suspended in a fluid and said fluid is contained in a vessel;
b) placing said vessel in a magnetic field gradient of sufficient strength to separate the magnetically labeled target entities from the non-target entities, wherein the magnetically labeled target entities are separated by moving to the walls of the vessel;
c) removing the fluid containing non-target entities from the vessel after the magnetically labeled entities are separated, the vessel being maintained in the magnetic field gradient;
d) adding fluid to the vessel being maintained in the magnetic field gradient and mixing; and
e) removing the fluid containing the non-target entities from the vessel being maintained in the magnetic field gradient after the magnetically labeled entities are separated.

2. The method according to claim 1, further comprising repeating steps d) and e) as required to obtain the desired purity of the target entities.

3. The method according to claim 1, further comprising collecting the fluid containing the non-target entities removed in steps c) and e).

4. The method according to claim 1, wherein the target entities are selected from the group consisting of cells, cell organelles, proteins and nucleic acids.

5. The method according to claim 4, wherein the target entities are cells.

6. The method according to claim 5, wherein the cells are selected from the group consisting of T cells, B cells, basophils, NK cells, dendritic cells, tumor cells, stem cells, hematopoietic progenitor cells, monocytes, mesenchymal cells, erythrocytes, mammary epithelial cells, eosinophils, neural cells, endothelial stem cells, endothelial progenitor cells and embryonic stem cells.

7. The method according to claim 1, wherein the vessel is selected from the group consisting of a tube, flask, plate, syringe, bottle, pipette and culture dish.

8. The method according to claim 7, wherein the tube is a centrifuge tube.

9. The method according to claim 1, wherein the sample is selected from the group consisting of whole blood, processed blood, tissue, bone marrow, pleural and peritoneal effusions, homogenized tumor sample and leukapheresis sample.

10. The method according to claim 9, wherein the sample is mouse spleen or density gradient separated human blood.

11. The method according to claim 9 or 10, wherein the sample is in the form of a cell suspension.

12. The method according to claim 1 wherein the magnetic particles have a mean diameter of less than 5 μm.

13. The method according to claim 1 wherein the magnetic particles have a mean diameter of less than 2 μm.

14. The method according to claim 1 wherein the magnetic particles have a mean diameter of less than 1 μm.

15. The method according to claim 1 wherein the magnetic particles have a mean diameter of less than 500 nm.

16. The method according to claim 1 wherein the magnetic particles have a mean diameter of about 160 nm.

17. The method according to claim 1 wherein the target entities are labeled with the magnetic particles using antibodies with specificity for the target entities.

18. The method according to claim 1 wherein the target entities are labeled with the magnetic particles using tetrameric antibody complexes.

19. The method according to claim 1, wherein the magnetic particles are selected from ferrofluids, other colloidal magnetic particles and particles in suspension.

20. The method according to claim 1, wherein the magnetic particles consist of a magnetic core.

21. The method according to claim 20, wherein the magnetic core is coated or embedded in material that prevents the particle cores from interacting.

22. The method according to claim 21, wherein the material that prevents the particle cores from interacting is selected from the group consisting of proteins, polysaccharides and synthetic polymers.

23. The method according to claim 21, wherein the material that prevents the particle cores from interacting is dextran.

24. The method according to claim 1, wherein the fluid is an aqueous solution.

25. The method according to claim 24, wherein the aqueous solution is saline or cell culture medium.

26. A method of automated magnetic separation of target entities from a sample containing a mixture of target entities and non-target entities suspended in a fluid using an automated instrument, with said method comprising:
a) automated labeling of said target entities with magnetic particles wherein said entities are suspended in a fluid;
b) automated placement of said fluid in a vessel in a magnetic field gradient of sufficient strength to separate the magnetically labeled target entities from the non-target entities, wherein the magnetically labeled target entities are separated by moving to the walls of the vessel;
c) automated removal of the fluid from the vessel after the magnetically labeled entities are separated, the vessel being maintained in the magnetic field gradient;
d) automated addition of fluid to the vessel being maintained in the magnetic field gradient and automated mixing; and
e) automated removal of the fluid from the vessel being maintained in the magnetic field gradient after the magnetically labeled entities are separated.

27. The method according to claim 26, further comprising repeating steps d) and e) as required to obtain the desired purity of the target entities.

28. The method according to claim 26, further comprising automated collection of the fluid containing the non-target entities removed in steps c) and e).

29. A method for treating malignant, non-malignant or genetic disorders comprising administering an effective amount of the target entities purified according to claim 1 to an animal in need thereof.

30. The method of claim 29, wherein the target entities are cells selected from the group consisting of T cells, B cells, basophils, NK cells, dendritic cells, tumor cells, stem cells, hematopoietic progenitor cells, monocytes, mesenchymal cells, erythrocytes, mammary epithelial cells, eosinophils, neural cells, endothelial stem cells, endothelial progenitor cells and embryonic stem cells.

31. A method for detecting cancer and metastatic disease in a biological sample comprising detecting the target entities purified by the method of claim 1.

32. A use of the method of claim 1 for generating a biological sample depleted of tumor cells.

33. A use of the method of claim 1 for generating a biological sample depleted of T-cells.

34. The use of claim 33, wherein the sample is selected from the group consisting of bone marrow, solid tumors, allogeneic grafts and autologous transplant tissue.
